(19) United States
(12) Patent Application Publication
(10) Pub. No.: US 2014/0315829 A1
(43) Pub. Date: Oct. 23, 2014

(54) Compositions And Methods For Enhancing Sialic Acid Levels In Tissue

(71) Applicant: The Regents of the University Of California, Oakland, CA (US)

(72) Inventors: Ajit Varki, La Jolla, CA (US); Kalyan Banda, San Diego, CA (US); Christopher J. Gregg, Roslindale, MA (US)

(73) Assignee: The Regents of the University Of California

(21) Appl. No.: 14/322,416

(22) Filed: Jul. 2, 2014

Related U.S. Application Data
(63) Continuation of application No. 13/973,185, filed on Aug. 22, 2013.
(60) Provisional application No. 61/691,993, filed on Aug. 22, 2012.

(51) Int. Cl. A61K 38/17; A61K 36/899

(52) U.S. Cl. CPC A61K 38/1741; A61K 38/1735; A61K 36/899
USPC 514/20.9

(57) ABSTRACT

The invention relates to compositions and methods for increasing sialic acid uptake and/or incorporation into tissue following gastrointestinal ingestion of compositions that contain sialic acid.

This application claims priority to co-pending U.S. provisional Application Ser. No. 61/691,993, filed on Aug. 22, 2012, which is herein incorporated by reference.

### GOVERNMENT INTEREST

This invention was made with government support under Grant No. GM032373 awarded by the National Institutes for Health (NIH). The government has certain rights in the invention.

### FIELD OF THE INVENTION

The invention relates to compositions and methods for increasing sialic acid uptake and/or incorporation into tissue following gastrointestinal ingestion of compositions that contain sialic acid.

### BACKGROUND OF THE INVENTION

All cells are covered with a dense and complex array of sugar chains that contain sialic acids (Sias) at the outermost units of these chains. By virtue of their terminal position, sialic acids act as binding sites for many exogenous and endogenous receptors such as the Influenza viruses and the Sialic family of endogenous proteins. Such sugars are thus useful drug targets for the prevention and treatment of infection. They are also involved in various biological and pathological processes such as neuronal plasticity and cancer metastasis. Sialic acids can be taken up from certain dietary sources (red meat and dairy products), and may also be associated with certain disease states, such as cancer and heart disease.

Sialic acids may contain N-acetyl groups and/or N-glycolyl groups. Mammals express two major sialic acids, N-acetylneuraminic acid and N-glycolylneuraminic acid (Neu5Gc). Although humans cannot produce Neu5Gc, it is detected in the epithelial lining of hollow organs, endothelial lining of the vasculature, fetal tissues, and carcinomas. This accumulation has relevance for diseases associated with such nutrients, via interaction with Neu5Gc-specific antibodies.

Mammalian infants require dietary sialic acid supplementation for optimal brain development (32). Dietary sialic acid also improves memory formation, learning metrics, and brain sialic acid content in piglets (33) and rats (34). Moreover, evidence has shown that breast milk as opposed to formula is much richer in sialic acid content (35, 36) and that breastfed children develop higher IQ levels than formula-fed children (37).

Despite these observations, remarkably little is known about the fate of ingested sialic acids in mammals. Aside from a few observations of sialidase activity in intestinal fluids (38), the only published studies on this topic were performed by Nöhle and Schauer (39-41). They showed that although radiolabeled free sialic acid fed to mice and rats appeared largely intact in the urine (39, 40), radiolabeled bound sialylated mucin-type glycoproteins were absorbed more slowly. A portion of the radioactive sialic acids were also metabolized (presumably by lyases), as evinced by radioactive CO₂ expired by the animals (41). Beyond this, little is known about the fate of ingested (free or bound) sialic acids in mammals.

In view of the importance of sialic acids in biological and pathological processes, such as binding sites for exogenous and endogenous receptors, microbial infection, neuronal plasticity, cancer, metastasis, and heart disease, what is needed are methods and compositions for modifying (e.g., increasing or decreasing) the level of sialic acids in tissue, such as the pool of sialic acids ingested by mammals, particularly humans.

### SUMMARY OF THE INVENTION

The invention provides a method for increasing the level of sialic acid in or on the cells of one or more peripheral tissues of a mammalian subject, comprising orally administering to the mammalian subject the combination of a) at least one purified sialic acid-glycoproteins, and b) one or more lipids, wherein administration is substantially simultaneous. In one embodiment, administration is to a fasted subject. In another embodiment, the peripheral tissue comprises blood, and the increase in the level of the sialic acid is from 1-fold to 500-fold. In another embodiment, the peripheral tissue comprises liver tissue, and the increase in the level of the sialic acid is from 1-fold to 50-fold. In a further embodiment, method further comprises measuring the level of the sialic acid in the peripheral tissue. In yet a further embodiment, the sialic acid comprises N-glycolylneuraminic acid (Neu5Gc). In another embodiment, the amount of the sialic acid-glycoprotein comprises from 0.1 to 1000 milligram sialic acid per kilogram (kg) body weight of the subject. In another embodiment, the lipid comprises from 0.1 to 100 milliliter per kg body weight. In one embodiment, the peripheral tissue comprises at least one of blood tissue and liver tissue. In a further embodiment, the mammalian subject is human. In one embodiment, the one or more lipids is selected from the group consisting of corn oil, olive oil, grape seed oil, soy bean oil, coconut oil and nut butters. In particular embodiment, the one or more lipids consists of corn oil.

The invention also provides an composition comprising a) at least one purified sialic acid-glycoproteins, and b) one or more lipids. In one embodiment, the one or more lipids is selected from the group consisting of corn oil, olive oil, grape seed oil, soy bean oil, coconut oil and nut butters. In particular embodiment, the one or more lipids consists of corn oil.

### BRIEF DESCRIPTION OF THE DRAWINGS

FIG. 1: 1 mg sialic acid-glycoprotein (porcine submaxillary mucin) (equivalent to 40 mg NeuGc/kg body weight) dissolved either in water or corn oil was fed to fasted and non-fasted Cmah−/− mice, followed by determining the level of N-glycolylneuraminic acid (Neu5Gc) recovered in the plasma (A) and in the livers (B) of Cmah−/− mice fasted overnight and then gavaged with 1 mg sialic acid-glycoprotein dissolved in corn oil using gavage needles, and non fasted Cmah−/− mice gavaged with the same concentration of 1 mg of the sialic acid-glycoprotein in water.

FIG. 2: Levels of Neu5Gc remaining in stomach and small intestinal contents 2 hours after feeding of the mice described in FIG. 1.

FIG. 3 shows stomachs of mice described in FIG. 1, that were fasted and gavaged with a solution of sialic acid glycoprotein dissolved in corn oil (bottom) were larger and retained more dye than stomachs of mice that were non fasted and gavaged with an aqeuous solution of sialic acid glycoprotein (top), at the end of 2 hours.

FIG. 4 (A) shows is the prior art structure of the nine-carbon backbone common to all known sialic acids shown, in the α configuration The following variations can occur at the carbon positions indicated: R1=H (on dissociation at physiological pH, gives the negative charge of Sia); can form lactones with hydroxyl groups on the same molecule or on other glycans; can form lactams with a free amino group at C-5; tauryl group. R2=H; alpha linkage to Gal(3/4/6), GalNAc(6), GlcNAc(4/6), Sia (8/9), or 5-O-Neu5Gc; oxygen linked to C-7 in 2,7-anhydro molecule; anomeric hydroxyl eliminated in Neu2en5Ac (double bond to C-3). R4=H; -acetyl; anhydro to C-8; Fuc; Gal. R5=Amino; N-acetyl; N-glycolyl; hydroxyl; N-acetimidoyl; N-glycolyl-O-acetyl; N-glycolyl-O-methyl; N-glycolyl-O-2-Neu5Gc. R7=H; -acetyl; anhydro to C-2; substituted by amino and N-acetyl in Leg. R8=H; -acetyl; anhydro to C-4; -methyl; -sulfate; Sia; Glc. R9=—H; -acetyl; -lactyl; -phosphate; -sulfate; Sia; OH substituted by H in Leg. (see Essentials of Glycobiology. 2nd edition. Chapter 14, Sialic Acids. Varki A, Cummings R D, Esko J D, et al., editors. Cold Spring Harbor (N.Y.): Cold Spring Harbor Laboratory Press; 2009). (B) shows the prior art structure of N-Glycolylneuraminic acid (Neu5Gc).

### DEFINITIONS

To facilitate understanding of the invention, a number of terms are defined below.

“Cytidine monophosphate-N-acetylneuraminic acid hydroxylase” and “CMAH” interchangeably refer to an enzyme that converts the sialic acid N-acetylneuraminic acid (Neu5Ac) to N-glycolylneuraminic acid (Neu5Gc.) In non-human mammals, Neu5Gc is recognized by a number of endogenous binding proteins, as well as by pathogenic organisms such as bacteria and viruses. Humans are unable to produce endogenous Neu5Gc because of an evolutionary inactivating mutation in their CMAH gene and are the only known mammals missing a functional CMAH gene (Chou, H-H, et al., Proc. Nat. Acad. Sci. (2002), 99(18): 11736-11741.) Although the cause for this mutation is unknown, it may have been caused by negative selection of individuals that were CMAH+, because of the recognition of Neu5Gc by pathogens.

The term “lack expression of cytidine monophosphate-N-acetylneuraminic acid hydroxylase (CMAH)” when in reference to a cell, tissue, organ, and/or organism means the substantial absence of expression of an enzymatically active cytidine monophosphate-N-acetylneuraminic acid hydroxylase (CMAH) and/or substantial absence of expression of mRNA that encodes an enzymatically active cytidine monophosphate-N-acetylneuraminic acid hydroxylase (CMAH).

“Fasting subject” refers to a subject that has abstained from ingesting into its gastrointestinal tract solids, liquids, etc., with the exception of water, for a period of at least 1 hour, more preferably up to 6 hours, up to 12 hours, up to 24 hours up to 48 hours, from 2 to 48 hours, from 2 to 24 hours, from 6 to 24 hours, from 6 to 12 hours, and/or from 2 to 12 hours.

“Tissue” refers to an aggregation of similarly specialized cells which together perform certain special functions in the body, and exemplified by muscle tissue, nerve tissue, epithelial tissue, and connective tissue.

“Peripheral tissue” refers to any tissue other than gastrointestinal tract tissue of the esophagus, stomach, small intestine, large intestine, and rectum. Thus, peripheral tissue includes heart, lung, brain, liver, basal ganglia, brain stem medulla, midbrain, pons, cerebellum, cerebral cortex, connective tissue, hypothalamus, eye, muscle, pituitary, thyroid, parathyroid, esophagus, thymus, adrenal glands, appendix, bladder, gallbladder, kidney, pancreas, spleen, skin (epithelial, etc), prostate, testes, ovaries, or uterus, any organ tissue, bone, flowing tissues such as blood and or lymph, and the like.

“Substantially simultaneous” in reference to oral administration, ingestion and/or feeding of sialic acid-glycoprotein compositions means that one component of the composition is administered at or near the same time as any other component of the composition. For example, where compositions of the present invention comprise a sialic-acid containing glycoprotein and at least one lipid, the lipid is ingested from zero (i.e., at the same time) to about 120 minutes before ingestion of the sialic acid-glycoprotein, such as from zero (i.e., at the same time) to about 60 minutes before ingestion of the sialic acid-glycoprotein and lipid. In particularly preferred embodiments, the sialic acid-glycoprotein is administered after the lipid to prevent the glycoprotein from passing into the intestine before the lipid component has exerted its beneficial effects

“Purify” and grammatical equivalents thereof when in reference to a desirable component (such as cell, protein, nucleic acid sequence, carbohydrate, sialic acid-glycoprotein, etc.) refer to the reduction in the amount of at least one undesirable component (such as cell, protein, nucleic acid sequence, carbohydrate, sialic acid-glycoprotein etc.) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in “enrichment” (i.e., an increase) in the amount of the desirable component relative to one or more undesirable component.

In some embodiments, a purified component may be “isolated,” for example if it is chemically cleaved from a native element, molecule, or structure and/or is chemically synthesized such that the “isolated” component is one that does not exist as in nature, and/or has a distinctive chemical identity from that of the native element, molecule, or structure to make the component markedly different from the one that exists in nature.

“Edible” means suitable to be ingested into the “gastrointestinal tract” (i.e., esophagus, stomach, small intestine, large intestine, and rectum) of an animal. For example, an edible composition does not cause substantial adverse effects (such as tissue erosion, vomiting, etc.) on the gastrointestinal tract of the animal ingesting it. In one embodiment, the lipid and/or sialic acid-glycoproteins of the invention's methods and compositions are edible.

“Sialic acid-glycoprotein” and “sialoglycoprotein,” “sialoglycopeptide,” and “polysialoglycoprotein,” interchangeably refer to a glycoprotein that contains at least one sialic acid moiety or derivative. Sialic acid-glycoproteins are exemplified by submaxillary mucins, salivary mucins, blood serum glycoproteins, fibrinogen, alpha-1-antitrypsin, antibodies, members of the major histocompatability complex (MHC), integrins, connective tissue proteins, and any protein capable of being glycosylated or modified with a sialic acid residue. Sialic acid-glycoproteins may be obtained from any species, such as from the mammalian species of pig, horse, goat, sheep, cow, and other livestock, and such from avian species, exemplified by the swallow, etc. Methods for preparing sialic acid-glycoproteins are known in the art. For example, mucins may be purified and/or isolated by precipitation in acidic pH. Exemplary methods for making submaxillary mucin are described herein (Example 1). Serum glycoproteins or other proteins may also be produced recombinantly, and also purified and/or isolated from the cellular component of blood.

The terms “increase,” “elevate,” “raise,” and grammatical equivalents (including “higher,” “greater,” etc.) when in reference to the level of any molecule (e.g., sialic acid, glycoprotein, sialic acid-glycoprotein, amino acid sequence, nucleic acid sequence, antibody, etc.), cell, and/or phenomenon (e.g., level of expression of a gene, disease symptom, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first sample (or the first subject) is exemplified by, but not limited to, a sample (or subject) that has been manipulated using the invention's compositions and/or methods. In a further embodiment, the second sample (or the second subject) is exemplified by, but not limited to, a sample (or subject) that has not been manipulated using the invention's compositions and/or methods. In an alternative embodiment, the second sample (or the second subject) is exemplified by, but not limited to, a sample (or subject) that has been manipulated, using the invention's compositions and/or methods, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the invention's compositions and/or methods is sought to be determined on one sample (or subject). In another embodiment, the first and second samples (or subjects) may be different, such as when comparing the effect of the invention's compositions and/or methods on one sample (subject), for example a patient participating in a clinical trial and another individual in a hospital.

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### DETAILED DESCRIPTION OF THE INVENTION

The state of understanding of how sialic acid-glycoproteins are digested by the GI tract is quite limited. Although dietary supplementation of free sialic acids has been shown to improve mammalian brain and immune system development in infants, it is not understood how to optimally deliver sialic acid orally to maximize these and other potential benefits.

In particular, feeding paradigms to specifically enhance uptake of dietary sialic acids have not been studied in the past. Recent published studies by our group have confirmed and extended prior data showing better uptake, delivery and metabolic incorporation of sialic acids glycosidically-linked to dietary glycoproteins (hereafter called “sialic acid-glycoproteins”), compared to dietary free sialic acids. Expanding on this observation, we compared fasting and non-fasting states and used various additives to the glycoprotein. We showed that the best results for improved uptake of bound sialic acids were achieved with fasting and addition of corn oil.

The invention's methods greatly enhance the delivery of dietary sialic acids from the gastrointestinal (GI) tract to the blood and other peripheral tissues. This invention describes feeding paradigms and techniques that have been shown experimentally to increase concentration of circulating levels of glycosidically bound sialic acids, as well as increased delivery to tissues.

Previous studies have shown that humans are unusual among mammals studied to date, in that they cannot synthesize the sialic acid Neu5Gc. However, this “non-human” sialic acid is incorporated in human tissues from our diet, with a circulating antibody response leading to a chronic inflammatory condition that can aggravate human carcinomas, atherosclerosis and susceptibility to some infectious diseases. It has also been shown experimentally that the sialic acid profile of cultured human cells can be altered by simply incubating these cells with another sialic acid. For example, a human cell culture that contains the “non-human” sialic acid, Neu5Gc, is a good model for the human condition wherein our cells have incorporated dietary Neu5Gc over our lifetime. Incubation of this culture with the “human” sialic acid, N-acetylneuraminic acid (Neu5Ac) rapidly replaces Neu5Gc with Neu5Ac, leading to a “washout” of Neu5Gc from the cells. Exploiting this, the Neu5Gc incorporated in our tissues could be subjected to a gradual “washout” by dietary Neu5Ac, if the circulating concentration of dietary Neu5Ac is high enough and/or efficient enough to lead to incorporation in the tissues.

The present invention is the first method described that can increase GI delivery of dietary sialic acids to peripheral tissues to facilitate these ends. In this regard, it believed that dietary Neu5Ac has nutritional value in human health in general or under specific conditions (e.g. infancy, stress, genetic disorders of sialic acid metabolism, etc.). This invention is an important way to maximize delivery of dietary Neu5Ac to tissues, to study the impact on such situations.

While not intending to limit the invention to a particular mechanism, the data below demonstrate that altering gastro-intestinal kinetics by using the invention's feeding paradigms reproducibly increases uptake and/or delivery of dietary sialic acids to peripheral tissues.

The invention is further described below.

### Methods for Increasing Sialic Acid in Tissue

The invention provides methods for increasing sialic acid uptake and/or incorporation into tissue following gastrointestinal ingestion of compositions that contain sialic acid. In one embodiment, the invention provides a method for increasing the level of sialic acid in one or more peripheral tissue of a mammalian subject, the method comprising orally administering to a subject (a) a composition containing one or more sialic acid-glycoproteins in combination with and/or substantially simultaneously with b) one or more lipids, wherein the combination is administered in an an amount that is sufficient for increasing the level of the sialic acid in the peripheral tissue compared to the same sialic acid-glycoproteins administered in the absence of the lipid or administered in the fed state.

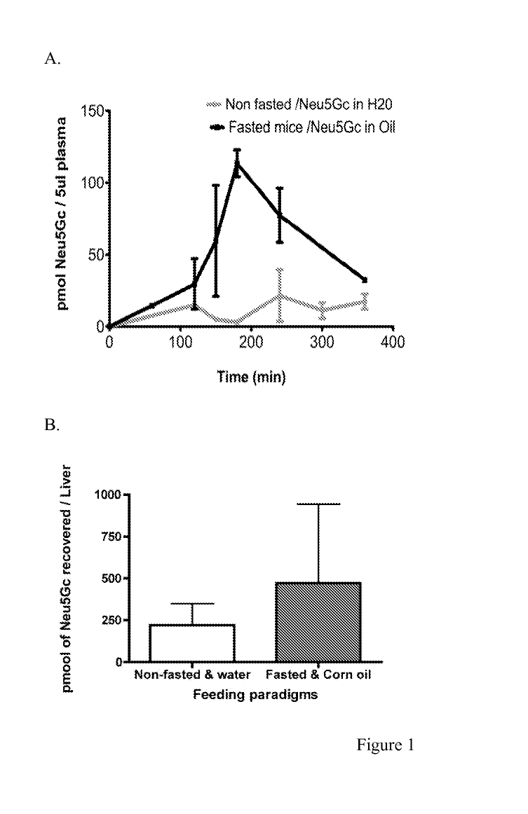

In one embodiment, the increase in the level of the sialic acid in the peripheral tissue is from 1-fold to 500-fold, from 1-fold to 400-fold, from 1-fold to 300-fold, from 1-fold to 200-fold, from 1-fold to 150-fold, from 1-fold to 100-fold, from 1-fold to 50-fold, from 1-fold to 40-fold, from 1-fold to 30-fold, from 1-fold to 20-fold, from 1-fold to 10-fold, from 1-fold to 5-fold, and most preferably from 1-fold to 4-fold. For example, data herein demonstrate from 5-fold to more than 100-fold increase in blood (serum) sialic acid concentration with fasting and use of corn oil in mice (FIG. 1B), and from 1-fold to 4-fold increase in the amount of sialic acid in the livers with fasting and use of corn oil in mice (FIG. 1C).

In one embodiment, it may be desirable to further measure the level of the sialic acid in the peripheral tissue, such as by Western blot, high performance liquid chromatography (HPLC), or combinations thereof (Example 1).

While not intending to limit the invention to a particular peripheral tissue in which the level of sialic acid is increased, in one embodiment, the peripheral tissue comprises at least one of blood tissue and liver tissue.

The invention's methods and compositions are useful in any mammalian “subject,” including humans, non-human primates, murines, ovines, bovines, ruminants, lagomorphs, porcines, caprines, equines, canines, felines, ayes, and the like).

In one embodiment, the subject lacks expression of cytidine monophosphate-N-acetylneuraminic acid hydroxylase (CMAH), as exemplified by a human subject and a transgenic non-human knockout Cmah^{-/-} animal, as described in U.S. Pat. No. 8,232,448, issued on Jul. 31, 2012 to Varki et al, the contents of which are incorporated here by reference in its entirety.

In one embodiment, the subject is fasting. In another embodiment, the subject is fed (not fasting)

### Sialic Acid-Glycoprotein and Sialic Acid

The invention contemplates the use of any one or more sialic acid-glycoproteins (i.e., singly or in combination). Exemplary sialic acid-glycoproteins include, for example and without limitation, mucins such as submaxillary mucin and salivary mucin, blood serum glycoprotein, fibrinogen, and alpha-1-antitrypsin, antibodies, members of the major histocompatability complex (MHC), integrins, connective tissue proteins, and any protein capable of being glycosylated.

In one embodiment, the amount of the sialic acid-glycoprotein administered is from 0.1 to 1000, from 0.1 to 500, from 1 to 400, from 1 to 300, from 1 to 200, and/or from 1 to 100 milligram (mg) sialic acid per kilogram (kg) body weight of the subject. In one embodiment, the amount is 40 mg sialic acid/kg body weight.

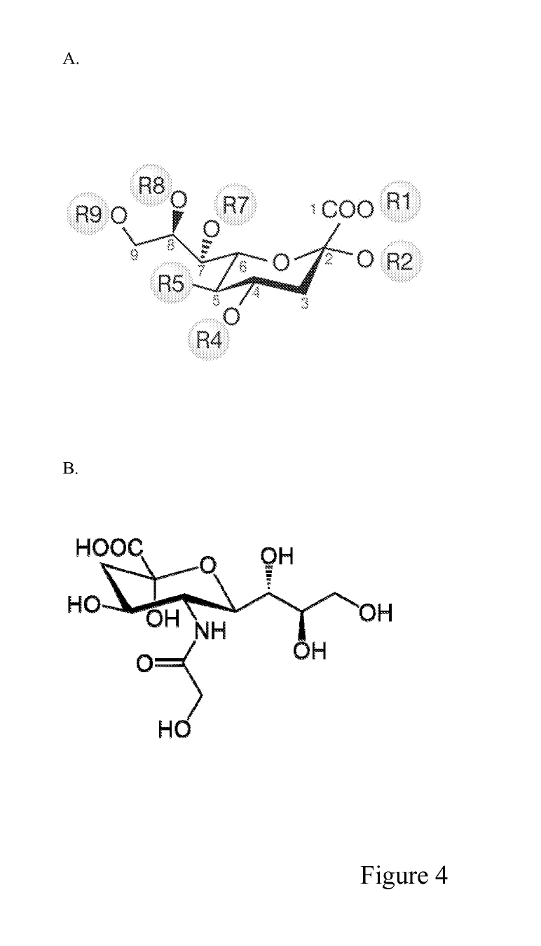

The sialic acid-glycoproteins that are useful in the invention's methods and compositions may contain any one or more sialic acid. “Sialic acid” and “Sia” interchangeably refers to a member of a family of “sialic acids” (also referred to as “Sias”) that describes the N-substituted derivatives and/or O-substituted derivatives of the deoxyamino sugar neuraminic acid, a monosaccharide with a nine-carbon backbone, as shown in FIG. 4A.

Sialic acid-glycoproteins may be purified and/or unpurified and/or a combination thereof.

Sialic acids are exemplified by N-glycolylneuraminic acid (Neu5Gc) (FIG. 4B), N-acetylneuraminic acid (Neu5Ac), and 2-Keto-3-deoxynonic acid (Kdn). Free Neu5Gc may be purchased commercially (Inalco, San Luis Obispo, Calif.) or synthesized according to published methods (43).

Sialic acids are typically present at the outermost acidic capping sugars on glycan chains, found on the cell surface and secreted glycoconjugates in animals of the Deuterostome lineage (vertebrates and so-called “higher” invertebrates (1-3)). The localization and ubiquity of sialic acids underscore their importance in mediating numerous cellular and extracellular interactions and their requirement for embryogenesis (4). The 9-carbon core structure of sialic acids can be extensively modified to fine-tune these interactions. For example, hydroxylation of the C5 N-acetyl group of CMP-Nacetylneuraminic acid (CMP-Neu5Ac) is catalyzed by the enzyme cytidine monophosphate N-acetylneuraminic acid hydroxylase, which generates CMP-N-glycolylneuraminic acid (CMP-Neu5Gc) (5-11). These two nucleotide sugars donate Neu5Ac and Neu5Gc for sialylation as the major sialic acids expressed in most mammals. A more detailed discussion regarding cytosolic pathways of sialic acid metabolism and the relevance to the intracellular fate of Neu5Gc is provided in Bergfeld, et al. (12). Interestingly, intracellular sialic acid biosynthetic enzymes do not discriminate between Neu5Gc and Neu5Ac, and exogenous Neu5Gc can exploit this metabolic “loophole” to be used for sialylation of molecules in human cells (18). This finding is important in view of the publication illustrating the presence of Neu5Gc in human carcinomas and fetal tissues (19) using anti-Neu5Gc antibodies generated in chickens (the avian lineage also appears deficient in Neu5Gc and thus can be immunized against the antigen) (20). More recently, histology of human tissues using an affinity-purified monospecific version of such an antibody (α-Neu5Gc IgY) has demonstrated the presence of Neu5Gc is in several human tissues, such as endothelial cells lining the micro- and macro-vasculature (21), carcinomas (22), placental tissues (23), and epithelial cells lining hollow organs (24, 25).

Given that humans who eat red meats and other mammal-derived food products consume milligram quantities of Neu5Gc each day (24), it is reasonable to propose that the Neu5Gc detected in human tissues originates from dietary sources. Previous human volunteer studies showed that orally ingested free Neu5Gc might be incorporated into salivary mucins in small amounts (24). However, the efficiency of incorporation was poor, and consequently no conclusions could be drawn regarding incorporation into endothelia, epithelia, cancers, or fetuses.

Although human sialic acid biosynthetic enzymes do not clearly discriminate between Neu5Ac and Neu5Gc, the human humoral immune system does, and all humans tested have circulating Neu5Gc-specific immunoglobulin (Ig) at variable (sometime high) levels (24, 26-28). These antibodies are known to arise during the 1st year of life (29). Recent work has also explored the potential pathologic role of Neu5Gc in human carcinomas (22, 30), atherosclerosis (21), and susceptibility to an Escherichia coli shiga-like SubAB toxin (25, 31). These studies suggest that Neu5Gc is actively exacerbating these diseases, in most cases through interactions with circulating Neu5Gc specific Ig (21, 22).

Thus, there is a need to understand mechanisms underlying tissue incorporation of ingested Neu5Gc to conclusively prove that dietary Neu5Gc can be accumulated in a manner mimicking human-like tissue distribution, and to consider enhanced uptake of dietary Neu5Ac could be used to “flush out” unwanted Neu5Gc by metabolic competition.

### Retention Vehicles: Oils, Fats and Lipids

The invention's methods and compositions contemplate using one or more lipid, oil, fat or other delivery vehicle. “Lipid” refers a hydrophobic and/or amphiphilic small molecule, such as fat and sterol-containing metabolites such as cholesterol. According to the present invention, lipids broadly embrace both fats and oils. Lipid may be liquid or solid at ambient room temperature. “Fat” refers to triglycerides, triesters of glycerol and fatty acids. Fats may be saturated, unsaturated, or a combination thereof. Fats may be either solid or liquid at room temperature, depending on their structure and composition. Fats include “oils” which refers to fats that are liquids at ambient room temperature, while “fat” is usually used to refer to fats that are solids at ambient room temperature. “Oil” is also used to refer to any neutral chemical substance that is a viscous liquid at ambient temperatures, is immiscible with water but soluble in alcohols or ethers. Oils have a high carbon and hydrogen content and are usually flammable and slippery (nonpolar). Fat includes organic fat, i.e., a fat produced by a plant, animal, and/or other organism through natural metabolic processes. Exemplary fats include, without limitation, vegetable fats (such as corn oil, olive oil, grape seed oil, soy bean oil, coconut oil, nut butters, etc.), and animal fats (such as fish oil, butter, suet, lard, whale blubber, etc.). While not intending to limit the amount of lipid used in the invention's methods and compositions, in one embodiment, the lipid comprises from 0.1 to 100, from 0.1 to 50, from 1 to 50, and/or from 1 to 25 mL per kg body weight of the subject ingesting it. In a preferred embodiment, the lipid is administered in an amount from 4 to 12 milliliter per kg body weight.

### Compositions

The invention provides kits, compositions and combinations for increasing sialic acid uptake and/or incorporation into peripheral tissue following oral ingestion of composition or combination that contains sialic acid, whether free sialic acid or sialic acid glycoproteins. In one embodiment, the invention provides a composition that comprises a) one or more purified sialic acid-glycoprotein, and b) one or more lipids.

### EXPERIMENTAL

The following examples serve to illustrate certain preferred embodiments and aspects of the present invention and are not to be construed as limiting the scope thereof

### Example 1

### Materials and Methods

### Neu5Gc Glycoproteins for Feeding Studies

Despite the ubiquity of Neu5Gc in most mammals, CMPNeu5Ac hydroxylase is nonfunctional in all humans (13, 14) due to an Alu-mediated deletion of CMAH exon 6 (15), causing premature truncation of the open reading frame (14). Thus, humans cannot produce Neu5Gc, only Neu5Ac. Neu5Gc is also absent in human-like Cmah^{-/-} mouse (16, 17), showing that there exists no alternative pathway for Neu5Gc biosynthesis in mammals. For this reason, studies to trace the delivery of sialic acid-containing glycoproteins were conducted in the Cmah^{-/-} mice and by tracing the delivery of Neu5Gc. It is expected that the feeding paradigms described herein will produce the same effect for other sialic acid moieties such as Nue5Ac.

Porcine submaxillary mucin (44) was used as a source of mucin-type glycosidically linked Neu5Gc-containing glycoproteins (Neu5Gc glycoproteins). Porcine submaxillary glands (Pel-Freez Biologicals, Rogers, Ark.) were finely chopped and homogenized in 5 volumes of water. Homogenates were centrifuged at 8000 rcf for 15 min, and the supernatant was then filtered through glass wool. The mucin was precipitated by gradual acidification (to pH 3.5) at 4° C., mixed overnight at 4° C., and then left to settle. The supernatant was removed by siphoning, and the precipitated mucin was centrifuged at 400 rcf for 15 min, washed with water, and centrifuged again. Mucin pellets were neutralized to pH 8.0 and dialyzed using a 10,000 molecular weight cutoff CE membrane (Spectrum Labs) against 20 volumes of water, with at least 5 volume changes. This preparation, called porcine submaxillary mucin (PSM), was then dried by lyophilization, and its Neu5Gc content was characterized by DMB-HPLC. PSM was chosen for this work because the only previous dietary feeding studies of sialic acid used a radioactively resialylated mucin (39, 40) and because it has very high Neu5Gc content (7-9% by weight). Neu5Gc content was less than 1% by weight of the chow. Neu5Gc-glycoprotein chow was generated by adding purified PSM to Neu5Gc-free chow, followed by autoclaving. Alternatively, purified PSM was provided to a manufacturer for incorporation into the chow prior to pelleting and sterilization by γ-irradiation. Neither autoclaving nor irradiation caused significant release of Neu5Gc from PSM. A Western blot of the chow was also run before and after sterilization and showed no change.

### Blood and Urine Kinetic Studies

Animals were gavaged, as above. The submandibular bleeding technique was used whereby blood is sampled from a conscious animal by puncturing the submandibular cheek pouch with a 5.0-mm lancet (Goldenrod Animal Lancets). Minimum blood volume (25-50 μl) was collected in plain glass capillary tubes and allowed to clot in serum microtainers (BD Biosciences). Serum was isolated by spinning tubes at 10,000 rcf for 2 min and stored at −20° C. Animals were bled at most three times. Urine was collected by restraining a conscious animal and taking advantage of spontaneous urination. If necessary, animals were gently massaged from the sternum in the caudal direction to induce urination. Urine was collected in plain capillary tubes and stored at −80° C. Quantification of Free and Glycosidically Linked Neu5Gc by DMB-HPLC—Neu5Gc in tissue, blood, and urine samples was measured by high performance liquid chromatography (HPLC) on a LaChrom Elite HPLC (Hitachi) by tagging sialic acids with the fluorogenic substrate, 1,2-diamino-4,5-methylene-dioxybenzene (DMB, Sigma), using previously described methods (23). HPLC runs were performed at 0.9 ml/min in 85% H₂O, 7% MeOH, 8% CH₃CN. Fluorescent signals were excited at 373 nm and acquired at 448 nm. Specific volumes of tissue homogenates were taken to maintain total sample sialic acid amounts below a 4-nmol threshold as follows: stomach/small/large intestinal wall samples (100 μl homogenate); stomach/small/large intestinal contents (100 μl homogenate); liver (20 μl homogenate); kidney (20 μl homogenate); serum (5 μl homogenate); urine (5 μl homogenate), and feces (100 μl homogenate). To quantify free sialic acids in these samples, homogenates were diluted and clarified by centrifugation at 10,000 rcf for 5 min at room temperature. Next, the supernatant was transferred to a Microcon-10, 10,000 molecular weight cutoff centrifugal filter (Millipore) and spun at 14,000 rcf for 15 min. The retentate was washed with 400 μl of H₂O and spun again. Free sialic acids in the run-through were dried down (Eppendorf Vacufuge), resuspended in H₂O, and derivatized with a 2_{—} DMB solution, which contained 7 mM DMB, 1.4 M acetic acid, 0.75 M β-mercaptoethanol, 18 mM sodium hydrosulfite. Samples were derivatized in the dark at 50° C. for 2.5 h. To quantify total sialic acids, sialic acids were first de-O-acetylated in 0.1 M NaOH for 30 min at 37° C. Next, glycosidically linked sialic acids were released by acid hydrolysis in 2 M acetic acid at 80° C. for 3 h. Samples were clarified, spun through a Microcon-10, washed, dried down, resuspended, and derivatized as above. Peak areas on HPLC were quantified by comparison with a standard curve of known Neu5Ac (Inalco Chemicals) and derivatized in parallel. Retention times of Neu5Gc (and Neu5Ac) in a given HPLC experiment were determined using chemically synthesized standards for Neu5Ac and Neu5Gc, as well as known biologic standards for O-acetylated sialic acids (purified bovine submaxillary mucin sialic acids), also derivatized in parallel.

### Detection of Neu5Gc by Western Blot

Tissue homogenates were lysed by boiling in sample buffer. The supernatant following centrifugation was loaded on 10% polyacrylamide mini gels (Bio-Rad), electrophoresed, and transferred to PVDF membranes (Bio-Rad) using a Fastblot semi-dry transfer system (Biometra). Mild periodate pretreatment of membranes to confirm specificity of anti-Neu5Gc signals was performed by quickly washing PVDF membranes three times in H₂O, washing three times in PBS, pH 6.5, for 5 min, then exposing membranes to freshly made 2 mM NaIO4 in PBS, pH 6.5, for 30 min in the dark at room temperature (or PBS control), then quickly washing membranes three times in H₂O, and finally washing membranes three times in H₂O for 5 min. Blocking, antibody incubations, and washes were then performed on the Snap-ID Vacuum Incubation System (Millipore). Membranes were blocked with 30 ml of 0.5% Neu5Gc-free cold water fish gelatin (Sigma) in Tris-buffered saline containing 0.1% Tween (TBST+FG). Membranes were then incubated with 3 ml of chicken Neu5Gc-specific antibody (αNeu5Gc IgY, Sialix, Inc.), diluted 1:25,000 in TBST, washed six times with 30 ml of TBST+FG, and then incubated with 3 ml of HRP-anti-chicken-IgY (Jackson ImmunoResearch), diluted 1:25,000. Signals were visualized by Immobilon chemiluminescence (Millipore), followed by exposure to Kodak BioMax XAR film for 5-30 s.

### Detection of Neu5Gc by Histology

Tissues from animals were either flash-frozen in OCT (Sakura) or fixed in 10% neutral buffered formalin for 24 h and then paraffin-embedded. In the case of the small intestinal segments, each segment was cut open lengthwise and rolled up from the proximal end to the distal end with the mucosal side facing outward. The rolls were fixed in 10% neutral buffered formalin for 24 h, then paraffinprocessed, and embedded. The rolls were sectioned at 5 μm, then deparaffinized in xylene, followed by rehydration in graded ethanol dilutions, and submersion in phosphate-buffered saline with 0.1% Tween (PBST). The slides were overlaid with blocking buffer (0.5% cold water fish gelatin in PBST) and blocked for endogenous biotin (Vector Laboratories, Burlingame, Calif.) and peroxidase. Slides were incubated overnight at 4° C. with the αNeu5Gc IgY (1:5000) and the control IgY (1:5000; Jackson ImmunoResearch). Slides were then washed and incubated with the biotinylated donkey anti-chicken IgY (1:500; Jackson ImmunoResearch) and then with Cy3-streptavidin (1:500; Jackson ImmunoResearch) for 30 min each. Cell nuclei were stained by incubation with DAPI (1:200,000; Sigma). Slides were then mounted in VectaMount (Vector Laboratories) and visualized by fluorescence microscopy. For cryopreserved liver and postnatal day 1 specimens, frozen sections were cut from the OCT blocks rehydrated in PBST. Next, the slides were blocked for nonspecific binding, blocked for endogenous biotin/peroxidase, and post-fixed in 10% neutral buffered formalin (Fisher). Slides were incubated with antibodies as above, except that the secondary antibody was followed by peroxidase/streptavidin (1:500; Jackson ImmunoResearch), developed with 3-amino-9-ethylcarbazole substrate (Vector Laboratories), and counterstained with Mayer's hematoxylin (Sigma). Slides were then mounted in Vecta-Mount (Vector Laboratories) and visualized by bright field microscopy.

### Example 2

### The Combination of Fasting and the Use of Corn Oil Increased Sialic Acid Levels in Blood and Peripheral Tissue

To enhance uptake fasted and non-fasted mice were gavaged with sialic acid-glycoprotein (porcine submaxillary mucin) using water and corn oil respectively. Cmah^{-/-} mice (described previously (16)) were either fasted overnight or allowed to eat ad-libitum and then gavaged with 1 mg sialic acid-glycoprotein (equivalent to 40 mg NeuGc/kg body weight) dissolved either in water or corn oil using gavage needles. Fed mice were gavaged with mg sialic acid-glycoprotein in a aqueous solution to mimic the control or normal fed state. Since Cmah null mice are devoid of Neu5Gc, dietary Neu5Gc can be used as a tracer to follow the uptake of sialic acids from the gut.

Using the submandibular bleeding technique, blood was sampled from the animals at various time points. Minimum blood volume (25-50 μL) was collected in plain glass capillary tubes and transferred to plasma microcontainers. Sialic acids derived from diet specifically, N-glycolylneuraminic acid (Neu5Gc) was measured in 5 μL of plasma by high performance liquid chromatography (HPLC) on a LaChrom Elite HPLC (Hitachi) by tagging sialic acids with the fluorogenic substrate, 1,2-diamino-4,5-methylene-dioxybenzene (DMB) using previously described methods.

The results shown in FIG. 1B (Table 1) revealed a several fold increase in circulating sialic acids with fasting and use of corn oil (6 mice were used to test each paradigm). Fasted mice with water and non-fasted mice with oil were also used in the experiment with no significant difference compared to both interventions together. Fasted mice also showed an earlier peak to maximum concentration at 200 minutes whereas fed mice had a peak concentration at around 240 minutes.

TABLE 1
Peak sialic acid glycoprotein in plasma

| Fed state | Fasted (pmol | | Fed (pmol | |
|---|---|---|---|---|
| | Neu5Gc/5 ul plasma) | | Neu5Gc/5 ul plasma) | |
| Delivery Mode | water | Oil | Water | Oil |
| Cmah mice (n = 6) | ~25 | ~120 | ~25 | ~25 |

Similarly, FIG. 1C (Table 2) shows that total dietary sialic acids recovered from the liver were greater showing increased delivery to peripheral tissues (2 mice each were used to test each aspect).

TABLE 2
Peak sialic acid glycoprotein in liver

| Fed state | Fasted | | Fed | |
|---|---|---|---|---|
| | (pmol Neu5Gc/liver) | | (pmol Neu5Gc/liver) | |
| Delivery Mode | water | Oil | Water | Oil |
| Cmah mice (n = 2) | ~250 | ~500 | ~250 | ~250 |

### Example 3

### Fasting and the Use of Corn Oil Increased Recovery of Dietary Sialic Acid from Peripheral Tissues

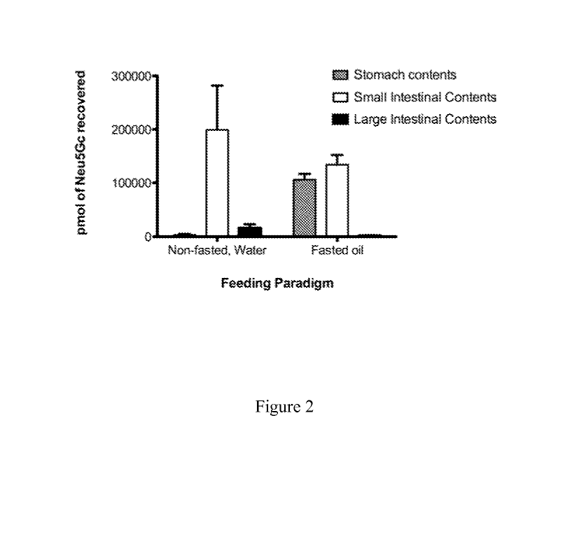

To understand why this feeding paradigm increased circulating and tissue levels we studied the GI kinetics of dietary sialic acid-glycoprotein uptake in the same mice. FIG. 2 shows that fasting and use of corn oil increase the amount of Neu5Gc remaining in stomach and small intestinal contents 2 hours after feeding (2 mice in each paradigm). The data reveal that more glycoprotein remains in the stomach in the combination feeding paradigm fasted+formulated in oil.

### Example 4

### Fasting and the Use of Oil Delays Gastric Emptying Time

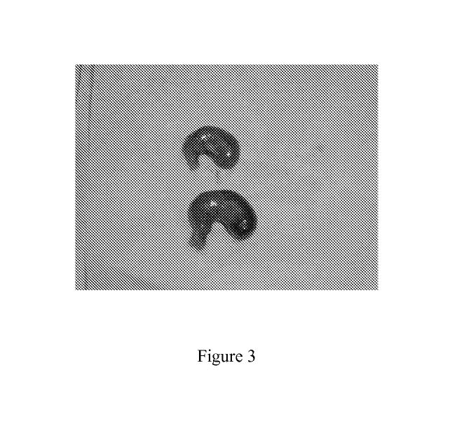

Experiments conducted using methylene blue dye administered using the same experimental feeding protocols as above (except that the blue dye replaced the dietary sialic acid-glycoprotein component) revealed that fasting and the use of corn oil delays gastric emptying time and decreases small and large intestinal transit time (FIG. 3). FIG. 3 shows stomachs of mice that were fasted and gavaged corn oil were larger and retained at least 50% more dye at the end of 2 hours indicating that gastric emptying time was delayed.

### EQUIVALENTS AND SCOPE

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

### REFERENCES

1. Schauer, R. (1978) Characterization of sialic acids. Methods Enzymol. 50, 64-89
2. Troy, F. A. (1992) Polysialylation. From bacteria to brains. Glycobiology 2, 5-23
3. Varki, A., and Schauer, R. (2009) in Essentials of Glycobiology (Varki, A., Cummings, R. D., Esko, J. D., Freeze, H. H., Stanley, P., Bertozzi, C. R., Hart, G. W., and Etzler, M. E., eds) pp. 199-218, Cold Spring Harbor Laboratory Press, Cold Spring Harbor, N.Y.
4. Schwarzkopf, M., Knobeloch, K. P., Rohde, E., Hinderlich, S., Wiechens, N., Lucka, L., Horak, I., Reutter, W., and Horstkorte, R. (2002) Sialylation is essential for early development in mice. Proc. Natl. Acad. Sci. U.S.A. 99, 5267-5270
5. Shaw, L., and Schauer, R. (1988) The biosynthesis of N-glycoloylneuraminic acid occurs by hydroxylation of the CMP-glycoside of N-acetylneuraminic acid. Biol. Chem. Hoppe-Seyler 369, 477-486
6. Shaw, L., Schneckenburger, P., Schlenzka, W., Carlsen, J., Christiansen, K., Jürgensen, D., and Schauer, R. (1994) CMP-N-acetylneuraminic acid hydroxylase from mouse liver and pig submandibular glands. Interaction with membrane-bound and soluble cytochrome b5-dependent electron transport chains. Eur. J. Biochem. 219, 1001-1011
7. Takematsu, H., Kawano, T., Koyama, S., Kozutsumi, Y., Suzuki, A., and Kawasaki, T. (1994) Reaction mechanism underlying CMP-N-acetylneuraminic acid hydroxylation in mouse liver. Formation of a ternary complex of cytochrome b5, CMP-N-acetylneuraminic acid, and a hydroxylation enzyme. J. Biochem. 115, 381-386
8. Kawano, T., Koyama, S., Takematsu, H., Kozutsumi, Y., Kawasaki, H., Kawashima, S., Kawasaki, T., and Suzuki, A. (1995) Molecular cloning of cytidine monophospho-N-acetylneuraminic acid hydroxylase. Regulation of species- and tissue-specific expression of N-glycolylneuraminic acid. J. Biol. Chem. 270, 16458-16463
9. Kelm, S., and Schauer, R. (1997) Sialic acids in molecular and cellular interactions. Int. Rev. Cytol. 175, 137-240
10. Angata, T., and Varki, A. (2002) Chemical diversity in the sialic acids and related _-keto acids. An evolutionary perspective. Chem. Rev. 102, 439-469
11. Varki, A. (2010) Colloquium paper. Uniquely human evolution of sialic acid genetics and biology. Proc. Natl. Acad. Sci. U.S.A. 107, 8939-8946
12. Bergfeld, A. K., Pearce, O. M., Diaz, S. L., Pham, T., and Varki, A. (2012) Metabolism of vertebrate amino sugars with N-glycolyl groups. Elucidating the intracellular fate of the non-human sialic acid N-glycolylneuraminic acid. J. Biol. Chem. 287, 28865-28881
13. Irie, A., Koyama, S., Kozutsumi, Y., Kawasaki, T., and Suzuki, A. (1998) The molecular basis for the absence of N-glycolylneuraminic acid in humans. J. Biol. Chem. 273, 15866-15871
14. Chou, H. H., Takematsu, H., Diaz, S., Iber, J., Nickerson, E., Wright, K. L., Muchmore, E. A., Nelson, D. L., Warren, S. T., and Varki, A. (1998) A mutation in human CMP-sialic acid hydroxylase occurred after the Homo-Pan divergence. Proc. Natl. Acad. Sci. U.S.A. 95, 11751-11756
15. Hayakawa, T., Satta, Y., Gagneux, P., Varki, A., and Takahata, N. (2001) Alu-mediated inactivation of the human CMP-N-acetylneuraminic acid hydroxylase gene. Proc. Natl. Acad. Sci. U.S.A. 98, 11399-11404
16. Hedlund, M., Tangvoranuntakul, P., Takematsu, H., Long, J. M., Housley, G. D., Kozutsumi, Y., Suzuki, A., Wynshaw-Boris, A., Ryan, A. F., Gallo, R. L., Varki, N., and Varki, A. (2007)N-Glycolylneuraminic acid deficiency in mice. Implications for human biology and evolution. Mol. Cell. Biol. 27, 4340-4346
17. Naito, Y., Takematsu, H., Koyama, S., Miyake, S., Yamamoto, H., Fujinawa, R., Sugai, M., Okuno, Y., Tsujimoto, G., Yamaji, T., Hashimoto, Y., balt3/zbc-bc/zbc-bc/zbc03412/zbc1803-12z ZSUBMIT 11 xppws S_{—}3 30/7/12 1:39 Itohara, S., Kawasaki, T., Suzuki, A., and Kozutsumi, Y. (2007) Germinal center marker GL7 probes activation-dependent repression of N-glycolylneuraminic acid, a sialic acid species involved in the negative modulation of B-cell activation. Mol. Cell. Biol. 27, 3008-3022
18. Bardor, M., Nguyen, D. H., Diaz, S., and Varki, A. (2005) Mechanism of uptake and incorporation of the non-human sialic acid N-glycolylneuraminic acid into human cells. J. Biol. Chem. 280, 4228-4237
19. Malykh, Y. N., Schauer, R., and Shaw, L. (2001) N-Glycolylneuraminic acid in human tumors. Biochimie 83, 623-634
20. Schauer, R., Srinivasan, G. V., Coddeville, B., Zanetta, J. P., and Guérardel, Y. (2009) Low incidence of N-glycolylneuraminic acid in birds and reptiles and its absence in the platypus. Carbohydr. Res. 344, 1494-1500
21. Pham, T., Gregg, C. J., Karp, F., Chow, R., Padler-Karavani, V., Cao, H., Chen, X., Witztum, J. L., Varki, N. M., and Varki, A. (2009) Evidence for a novel human-specific xeno-autoantibody response against vascular endothelium. Blood 114, 5225-5235
22. Hedlund, M., Padler-Karavani, V., Varki, N. M., and Varki, A. (2008) Evidence for a human-specific mechanism for diet and antibody-mediated inflammation in carcinoma progression. Proc. Natl. Acad. Sci. U.S.A. 105, 18936-18941
23. Diaz, S. L., Padler-Karavani, V., Ghaderi, D., Hurtado-Ziola, N., Yu, H., Chen, X., Brinkman-Van der Linden, E. C., Varki, A., and Varki, N. M. (2009) Sensitive and specific detection of the non-human sialic acid Nglycolylneuraminic acid in human tissues and biotherapeutic products. PLoS ONE 4, e4241
24. Tangvoranuntakul, P., Gagneux, P., Diaz, S., Bardor, M., Varki, N., Varki, A., and Muchmore, E. (2003) Human uptake and incorporation of an immunogenic nonhuman dietary sialic acid. Proc. Natl. Acad. Sci. U.S.A. 100, 12045-12050
25. Byres, E., Paton, A. W., Paton, J. C., Löfling, J. C., Smith, D. F., Wilce, M. C., Talbot, U. M., Chong, D. C., Yu, H., Huang, S., Chen, X., Varki, N. M., Varki, A., Rossjohn, J., and Beddoe, T. (2008) Incorporation of a nonhuman glycan mediates human susceptibility to a bacterial toxin. Nature 456, 648-652
26. Nguyen, D. H., Tangvoranuntakul, P., and Varki, A. (2005) Effects of natural human antibodies against a non-human sialic acid that metabolically incorporates into activated and malignant immune cells. J. Immunol. 175, 228-236
27. Padler-Karavani, V., Yu, H., Cao, H., Chokhawala, H., Karp, F., Varki, N., Chen, X., and Varki, A. (2008) Diversity in specificity, abundance, and composition of anti-Neu5Gc antibodies in normal humans. Potential implications for disease. Glycobiology 18, 818-830
28. Padler-Karavani, V., Hurtado-Ziola, N., Pu, M., Yu, H., Huang, S., Muthana, S., Chokhawala, H. A., Cao, H., Secrest, P., Friedmann-Morvinski, D., Singer, O., Ghaderi, D., Verma, I. M., Liu, Y. T., Messer, K., Chen, X., Varki, A., and Schwab, R. (2011) Human xeno-autoantibodies against a non-human sialic acid serve as novel serum biomarkers and immunotherapeutics in cancer. Cancer Res. 71, 3352-3363
29. Taylor, R. E., Gregg, C. J., Padler-Karavani, V., Ghaderi, D., Yu, H., Huang, S., Sorensen, R. U., Chen, X., Inostroza, J., Nizet, V., and Varki, A. (2010) Novel mechanism for the generation of human xeno-autoantibodies against the nonhuman sialic acid N-glycolylneuraminic acid. J. Exp. Med. 207, 1637-1646
30. Yin, J., Hashimoto, A., Izawa, M., Miyazaki, K., Chen, G. Y., Takematsu, H., Kozutsumi, Y., Suzuki, A., Furuhata, K., Cheng, F. L., Lin, C. H., Sato, C., Kitajima, K., and Kannagi, R. (2006) Hypoxic culture induces expression of sialin, a sialic acid transporter, and cancer-associated gangliosides containing non-human sialic acid on human cancer cells. Cancer Res. 66, 2937-2945
31. Löfling, J. C., Paton, A. W., Varki, N. M., Paton, J. C., and Varki, A. (2009) A dietary non-human sialic acid may facilitate hemolytic-uremic syndrome Kidney Int. 76, 140-144
32. Wang, B. (2009) Sialic acid is an essential nutrient for brain development and cognition. Annu. Rev. Nutr. 29, 177-222
33. Wang, B., Yu, B., Karim, M., Hu, H., Sun, Y., McGreevy, P., Petocz, P., Held, S., and Brand-Miller, J. (2007) Dietary sialic acid supplementation improves learning and memory in piglets. Am. J. Clin. Nutr. 85, 561-569
34. Bode, L. (2009) Human milk oligosaccharides. Prebiotics and beyond. Nutr. Rev. 67, S183-S191
35. Wang, B., Brand-Miller, J., McVeagh, P., and Petocz, P. (2001) Concentration and distribution of sialic acid in human milk and infant formulas. Am. J. Clin. Nutr. 74, 510-515
36. Morgan, B. L., and Winick, M. (1979) A possible relationship between brain N-acetylneuraminic acid content and behavior. Proc. Soc. Exp. Biol. Med. 161, 534-537
37. Anderson, J. W., Johnstone, B. M., and Remley, D. T. (1999) Breast-feeding and cognitive development. A meta-analysis. Am. J. Clin. Nutr. 70, 525-535
38. Dickson, J. J., and Messer, M. (1978) Intestinal neuraminidase activity of suckling rats and other mammals. Relationship to the sialic acid content of milk. Biochem. J. 170, 407-413
39. Nöhle, U., Beau, J. M., and Schauer, R. (1982) Uptake, metabolism, and excretion of orally and intravenously administered, double-labeled N-glycoloylneuraminic acid and single-labeled 2-deoxy-2,3-dehydro-N-acetylneuraminic acid in mouse and rat. Eur. J. Biochem. 126, 543-548
40. Nöhle, U., and Schauer, R. (1981) Uptake, metabolism, and excretion of orally and intravenously administered, 14C- and 3H-labeled N-acetylneuraminic acid mixture in the mouse and rat. Hoppe-Seyler's Z. Physiol. Chem. 362, 1495-1506
41. Nöhle, U., and Schauer, R. (1984) Metabolism of sialic acids from exogenously administered sialyllactose and mucin in mouse and rat. Hoppe-Seyler's Z. Physiol. Chem. 365, 1457-1467
42. Varki, A., and Diaz, S. (1984) The release and purification of sialic acids from glycoconjugates. Methods to minimize the loss and migration of O-acetyl groups. Anal. Biochem. 137, 236-247
43. Pearce, O. M., and Varki, A. (2010) Chemo-enzymatic synthesis of the carbohydrate antigen N-glycolylneuraminic acid from glucose. Carbohydr. Res. 345, 1225-1229
44. Gottschalk, A. (1960) The Chemistry and Biology of Sialic Acids and Related Substances, pp. 1-115, Cambridge University Press, Cambridge, UK
45. Tso, P., and Balint, J. A. (1986) Formation and transport of chylomicrons by enterocytes to the lymphatics. Am. J. Physiol. 250, G715-G26
46. Norgard, K. E., Han, H., Powell, L., Kriegler, M., Varki, A., and Varki, N. M. (1993) Enhanced interaction of L-selectin with the high endothelial venule ligand via selectively oxidized sialic acids. Proc. Natl. Acad. Sci. U.S.A. 90, 1068-1072
47. Hirabayashi, Y., Kasakura, H., Matsumoto, M., Higashi, H., Kato, S., Kasai, N., and Naiki, M. (1987) Specific expression of unusual GM2 ganglioside with Hanganutziu-Deicher antigen activity on human colon cancers. Jpn. J. Cancer Res. 78, 251-260
48. Davies, L., Pearce, O. M., Tessier, M., Assar, S., Smutova, V., Pajunen, M., Sumida, M., Sato, C., Kitajima, K., Finne, J., Gagneux, P., Pchejetsky, A., Woods, R., and Varki, A. (2012) Metabolism of vertebrate amino sugars with N-glycolyl groups. Resistance of _{—}2-8-linked N-glycolylneuraminic acid to enzymatic cleavage. J. Biol. Chem. 287, 28917-28931
49. Carlson, D. M. (1968) Structures and immunochemical properties of oligosaccharides isolated from pig submaxillary mucins. J. Biol. Chem. 243, 616-626
50. Eckhardt, A. E., Timpte, C. S., DeLuca, A. W., and Hill, R. L. (1997) The complete cDNA sequence and structural polymorphism of the polypeptide chain of porcine submaxillary mucin. J. Biol. Chem. 272, 33204-33210

1-15. (canceled)

16. A method of increasing the level of Neu5Ac in the serum of a fasting subject comprising administering to said fasting subject a pharmaceutical composition consisting of Neu5Ac-bound glycoprotein dissolved in corn oil, wherein said pharmaceutical composition is administered in an amount equivalent to 40 mg/kg Neu5Ac.

17. The method of claim 16, wherein said fasting subject is human.

18. The method of claim 7, wherein said Neu5Ac-bound glycoprotein comprises mucin.

19. The method of claim 8, wherein said corn oil is present in the pharmaceutical composition at a concentration of from 4 mL/kg to 12 mL/kg.

20. The method of claim 19, wherein said fasting subject has abstained from ingesting solids and/or liquids for a period of from 2 to 48 hours prior to administration.

21. The method of claim 20, wherein the level of Neu5Ac in the serum is increased from 1-fold to 200-fold.
